(19) United States
(12) Patent Application Publication
(10) Pub. No.: US 2025/0250599 A1
(43) Pub. Date: Aug. 7, 2025

(54) POLYPHOSPHATE KINASES (PPKs) FOR EFFICIENT REGENERATION OF GUANOSINE TRIPHOSPHATE (GTP) AND USE THEREOF

(71) Applicant: BEIJING UNIVERSITY OF CHEMICAL TECHNOLOGY, Chaoyang District Beijing (CN)

(72) Inventors: Luo LIU, Chaoyang District, Beijing (CN); Chenqi CAO, Chaoyang District, Beijing (CN); Linbo WU, Chaoyang District, Beijing (CN); Ruikai WANG, Chaoyang District, Beijing (CN); Chenyuan LIU, Chaoyang District, Beijing (CN); Lintong JIANG, Chaoyang District, Beijing (CN)

(21) Appl. No.: 18/917,329

(22) Filed: Oct. 16, 2024

(30) Foreign Application Priority Data
Feb. 5, 2024 (CN) 202410164536.0

(51) Int. Cl. C12P 19/38; C12N 9/12; C12P 19/02

(52) U.S. Cl. CPC C12P 19/38; C12N 9/1229; C12P 19/02; C12Y 207/04001

(57) ABSTRACT

Polyphosphate kinases (PPKs) for efficient regeneration of guanosine triphosphate (GTP) and use thereof are provided, belonging to the field of biotechnology. The PPKs expressed and obtained in Escherichia coli BL21(DE3) can efficiently regenerate the GTP using tripolyphosphate (tripolyP), tetrapolyP, and hexametaphosphate as phosphate donors. Use of the PPKs in regeneration of GTP as well as synthesis of guanosine diphosphate (GDP)-L-fucose or GDP-mannose and a derivative thereof (such as fucosyllactose) significantly improves synthesis sustainability and reduces a synthesis cost.

### CROSS REFERENCE TO RELATED APPLICATION

This patent application claims the benefit and priority of Chinese Patent Application No. 202410164536.0, filed with the China National Intellectual Property Administration on Feb. 5, 2024, the disclosure of which is incorporated by reference herein in its entirety as part of the present application.

### REFERENCE TO SEQUENCE LISTING

A computer readable XML file entitled “GWP20240604592_seqlist”, that was created on Sep. 18, 2024, with a file size of about 10,954 bytes, contains the sequence listing for this application, has been filed with this application, and is hereby incorporated by reference in its entirety.

### TECHNICAL FIELD

The present disclosure relates to polyphosphate kinases (PPKs) for efficient regeneration of guanosine triphosphate (GTP) and use thereof, belonging to the field of biotechnology.

### BACKGROUND

Nucleoside triphosphates (NTPs) are important molecules for intracellular energy transfer and storage, and participate in various biochemical reactions in vivo, thus playing an important role. NTP metabolism is well conserved among species, such that NTP salts are generally prepared by de novo synthesis. Guanosine triphosphate (GTP) is an energy carrier in converting succinate coenzyme A into succinate in the tricarboxylic acid cycle, and can be mutually converted into adenosine triphosphate (ATP). At the same time, GTP plays an important role in nerves, metabolism, and cardiovascular system. Currently, the GTP is synthesized by fermentation and enzymatic methods using a series of enzymes. Therefore, the GTP is still relatively expensive. GTP is generally degraded into guanosine diphosphate (GDP)/guanosine monophosphate (GMP) during the reaction. Therefore, it is of great significance to develop an enzyme that can convert GDP/GMP back into GTP.

There are two types of polyphosphate kinases (PPKs). The PPK (now named PPK1) originally isolated from Escherichia coli reversibly catalyzes the transfer of a terminal Pi residue of the ATP to a short-chain polyphosphate (polyP) to generate a long-chain polyP. PPK2 has no obvious sequence similarity to the PPK1, preferentially catalyzes polyP-driven nucleotide phosphorylation, and requires metal ions to stimulate activity. PPK2 family can be divided into three subfamilies (class I, class II, and class III). The class I and class II PPK2s catalyze the phosphorylation of nucleoside diphosphates (NDPs) and nucleoside monophosphates (NMPs), respectively, while the class III PPK2 catalyzes the phosphorylation of NMPs and NDPs. Different PPK2s have great differences in their preferences for catalyzing NDPs, and most PPKs are active only in the presence of long-chain polyP.

GDP-L-fucose is a nucleotide substance existing in the human body as well as an indispensable substance for life activities. In clinical applications, the GDP-L-fucose is used to treat certain diseases, such as chronic hepatitis, progressive muscular atrophy, and vision loss. In addition, the GDP-L-fucose is a synthetic precursor of fucosyllactose. Fucosyllactose, including 2′-fucosyllactose (2′-FL) and 3-fucosyllactose (3-FL), belongs to an immunologically-essential trisaccharide and is abundant in human milk oligosaccharides (HMOs). As an active ingredient in human milk, the fucosyllactose has important physiological functions such as regulating intestinal flora, promoting infant brain development, regulating immune response, inhibiting inflammation, and resisting pathogenic microbial infections. As a result, the fucosyllactose is widely used in infant formula milk powder, nutritional health products, and food additives. Currently, fucosyllactose is mainly obtained by constructing engineered bacteria and fermenting same, while there are almost no reports on in vitro enzymatic synthesis of the fucosyllactose.

At present, most of the research on NTP regeneration system is focused on the regeneration of ATP from ADP, and there are few reports on the regeneration of GTP. Therefore, the development of a novel PPK that can regenerate GTP from bulk polyP shows broad application prospects.

### SUMMARY

In view of the problem that GTP substrate has a wide range of uses but is expensive, the present disclosure provides PPKs for efficient regeneration of GTP and use thereof. These PPKs can regenerate the GTP using polyP as a substrate.

The present disclosure adopts the following technical solutions:

The present disclosure provides PPKs for efficient regeneration of GTP, where the PPKs have amino acid sequences being one selected from the group consisting of:
(1) amino acid sequences shown in SEQ ID NO: 1, SEQ ID NO: 2, and SEQ ID NO: 3; and
(2) amino acid sequences having a same function and obtained by subjecting amino acid sequences shown in SEQ ID NO: 1, SEQ ID NO: 2, and SEQ ID NO: 3 to modification including substitution, deletion, or addition of one or more amino acids.

The present disclosure further provides PPK genes, where the PPK genes encode the PPKs. Preferably, the PPK genes have nucleotide sequences shown in SEQ ID NO: 4, SEQ ID NO: 5, and SEQ ID NO: 6.

The present disclosure further provides a recombinant plasmid vector carrying the PPK genes. Preferably, the recombinant plasmid vector includes but is not limited to pET28a (+).

The present disclosure further provides a recombinant microorganism including the recombinant plasmid vector. Preferably, the recombinant microorganism is Escherichia coli BL21(DE3).

The present disclosure further provides use of the PPKs in regeneration of GTP as well as synthesis of GDP-L-fucose or GDP-mannose and a derivative thereof using a polyP.

In an example of the present disclosure, the polyP as a donor is preferably one selected from the group consisting of tripolyP, tetrapolyP, and hexametaphosphate.

In an example of the present disclosure, the GDP-L-fucose is synthesized by in vitro multi-enzyme cascade catalysis using a GDP-L-fucose synthesis-related enzyme, and the GDP-L-fucose and lactose are catalyzed to generate fucosyllactose using fucosyltransferase.

In an example of the present disclosure, the PPKs are used to regenerate the GTP and synthesize the GDP-L-fucose and the fucosyllactose.

In an example of the present disclosure, the GDP-L-fucose de novo synthesis-related enzyme is selected from the group consisting of polyphosphate-dependent glucomannokinase (PPGMK) derived from Arthrobacter species, phosphomannomutase (manB) derived from Streptomyces coelicolor, mannose-1-phosphate guanylytransferase (manC) derived from Escherichia coli, GDP-D-mannose-4,6-hydratase (gmd) derived from Pseudomonas aeruginosa PAO1, and GDP-L-fucose synthase (wcaG) derived from Helicobacter pylori.

In an example of the present disclosure, the GDP-L-fucose salvage synthesis-related enzyme is preferably L-fucokinase/GDP-L-fucose pyrophosphorylase (fkp) from Bacteroides fragilis.

In an example of the present disclosure, the fucosyltransferase is selected from the group consisting of α-1,2-fucosyltransferase and α-1,3-fucosyltransferase, and is preferably derived from Helicobacter pylori.

In an example of the present disclosure, the PPKs are used to regenerate the GTP and synthesize the GDP-L-fucose and the fucosyllactose. A reaction system of the synthesis includes 0.1 mg/mL to 2 mg/mL of a GDP-L-fucose de novo synthesis-related enzyme, 0.1 mg/mL to 2 mg/mL of the fucosyltransferase, 0.1 mg/mL to 2 mg/mL of the PPKs, 1 mM to 50 mM of mannose, 1 mM to 50 mM of lactose, 1 mM to 50 mM of the polyP, 1 mM to 10 mM of the GTP or GDP, and 1 mM to 50 mM of nicotinamide adenine dinucleotide phosphate (NADPH or NADP⁺); and the synthesis is conducted under a pH value of 6 to 10 at 20° C. to 40° C. for 1 h to 48 h.

In an example of the present disclosure, the PPKs are used to regenerate the GTP and synthesize the GDP-L-fucose and the fucosyllactose. A reaction system of the synthesis includes 0.1 mg/mL to 2 mg/mL of a GDP-L-fucose salvage synthesis-related enzyme, 0.1 mg/mL to 2 mg/mL of the fucosyltransferase, 0.1 mg/mL to 2 mg/mL of the PPKs, 1 mM to 50 mM of fucose, 1 mM to 50 mM of lactose, 1 mM to 50 mM of the polyP, 1 mM to 10 mM of the GTP or GDP, and 1 mM to 10 mM of adenosine triphosphate (ATP) or adenosine diphosphate (ADP); and the synthesis is conducted under a pH value of 6 to 10 at 20° C. to 40° C. for 1 h to 48 h.

The present disclosure has the following beneficial effects:

Currently, most of the research on NTP regeneration system is focused on the regeneration of ATP from ADP, and there are few reports on the regeneration of GTP. The present disclosure provides a method for regenerating GTP with low cost and high efficiency. The PPKs involved can efficiently regenerate GTP with bulk polyP as a raw material and have broad application prospects.

In the present disclosure, the PPKs are used for GTP regeneration, and GDP-L-fucose and fucosyllactose are synthesized in vitro by cascade synthesis of the GDP-L-fucose synthesis-related enzyme and fucosyltransferase. With 20 mM (6.84 g/L) of lactose and 2 mM of GTP, an in vitro one-pot reaction was conducted for 48 h, and a 2′-FL yield reaches 5.93 g/L. Green and efficient in vitro synthesis of the GDP-L-fucose and fucosyllactose is achieved. The PPKs have broad application prospects for regeneration of GTP as well as synthesis of GDP-L-fucose or GDP-mannose and a derivative thereof.

### BRIEF DESCRIPTION OF THE DRAWINGS

FIG. 1 shows routes for regenerating GTP with PPK and some use examples;

FIG. 2 shows SDS-PAGE electrophoresis of the purification of PPK proteins;

FIG. 3 shows high-performance liquid chromatography (HPLC) peak profiles of GTP and GDP standards;

FIG. 4 shows activities of purified PPK proteins;

FIG. 5 shows HPLC peak profiles of lactose and 2′-FL standards; and

FIG. 6 shows use of PPK to regenerate GTP for one-pot synthesis of 2′-FL in vitro.

### DETAILED DESCRIPTION OF THE EMBODIMENTS

The present disclosure is described in detail below with reference to the examples.

PPK-Rs from Rhodobacter sphaeroides, PPK-Ft from Francisella tularensis, and PPK-Pa from Pseudomonas aeruginosa have amino acid sequences shown in SEQ ID NO: 1, SEQ ID NO: 2, and SEQ ID NO: 3, respectively; and corresponding nucleotide sequences are shown in SEQ ID NO: 4, SEQ ID NO: 5, and SEQ ID NO: 6, respectively.

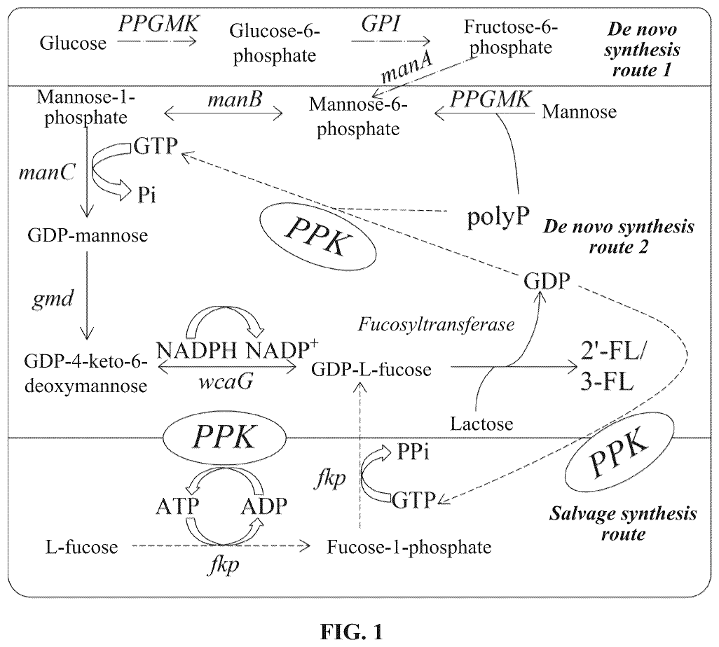

The route by which PPK utilizes polyP to regenerate GTP and cascade GDP-L-fucose synthesis-related enzyme and fucosyltransferase to efficiently synthesize GDP-L-fucose and fucosyllactose in vitro is shown in FIG. 1.

### Example 1: Construction, Expression, and Purification for Recombinant Bacterium of PPKs

### 1. Plasmid Construction and Transfection

3 PPK protein sequences were sent to GenScript for codon optimization and synthesis, where a plasmid vector used was pET-28a (+), and the restriction sites were NdeI and HindIII. The synthesized recombinant plasmid was transfected into Escherichia coli BL21(DE3), and single colonies were obtained after plating on plates for culture.

### 2. Protein Expression

Each of the single colonies were selected and inoculated into a culture tube containing 3 mL to 6 mL of LB medium (containing 50 μg/mL kanamycin) and cultured at 37° C. overnight. 1% to 2% of the bacterial solution was inoculated into 200 mL of a medium and cultured at 37° C. until OD600 was 0.6 to 0.8, and 0.2 mM IPTG was added to allow induction at low temperature of 20° C. under 180 rpm on a shaker for 16 h to 20 h. The bacterial cells were collected by centrifugation and resuspended in a 50 mM Tris-HCl buffer (pH=7.5-9). The cells were disrupted by ultrasonic cell disruptor and a supernatant was obtained by centrifugation.

### 3. Protein Purification

Since pET-28a (+) was used as the vector and NdeI and HindIII were restriction sites, the expressed protein with a His tag could be purified by a Ni-NTA affinity gravity column. An obtained supernatant was filtered through a 0.45 μm filter membrane and added into a column pre-equilibrated with an equilibrium buffer (50 mM Tris-HCl, 300 mM NaCl, pH=7.8-8.2). The miscellaneous proteins were washed with pre-cooled impurity washing buffer (50 mM Tris-HCl, 300 mM NaCl, 30 mM imidazole, pH=7.8-8.2) until a Coomassie Brilliant Blue detection solution did not turn blue. A pre-cooled elution buffer (50 mM Tris-HCl, 300 mM NaCl, 300 mM imidazole, pH=7.8-8.2) was added to elute a target protein. When an effluent turned blue with the Coomassie Brilliant Blue detection solution, the eluted protein was collected until the blue color was not too deep, so as to obtain a pure enzyme solution. After desalting through an ultrafiltration tube, the pure enzyme solution was used for the reactions in the subsequent examples.

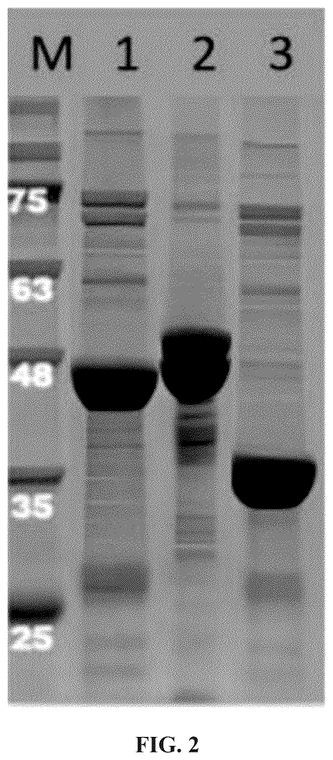

FIG. 2 showed SDS-PAGE electrophoresis of the purification of PPK proteins. In the figure, M represented a control marker (in kDa), Lane 1 represented purified PPK-Rs, Lane 2 represented purified PPK-Pa, and Lane 3 represented purified PPK-Ft. The theoretical molecular weights were as follows: PPK-Rs (40 kDa), PPK-Pa (42.9 kDa), and PPK-Ft (34.2 kDa). The electrophoresis results indicated that all 3 PPKs were purified well.

### Example 2: Activity Testing of PPK Purified Proteins

A reaction system of synthesis included: 5 mM MgCl₂, 5 mM polyP6 (sodium hexametaphosphate), 5 mM GDP, and purified enzyme solutions of different PPKs. A reaction was conducted at 30° C. for 1 h, and a product was detected by Shimadzu HPLC with a Diamonsil C18 column (5 μm, 250×4.6 mm), using a mobile phase A (20 mM potassium dihydrogen phosphate, 5 mM tetrabutylammonium hydrogen sulfate, 5% acetonitrile, pH=6.5) and a mobile phase B (acetonitrile). The gradient elution program included: mobile phase A was 90% at 0-5 min, decreased to 65% at 5-25 min, and increased to 90% at 25-30 min, with a flow rate of 1 mL/min, a UV detector at a wavelength of 254 nm, and an injection volume of 10 μL.

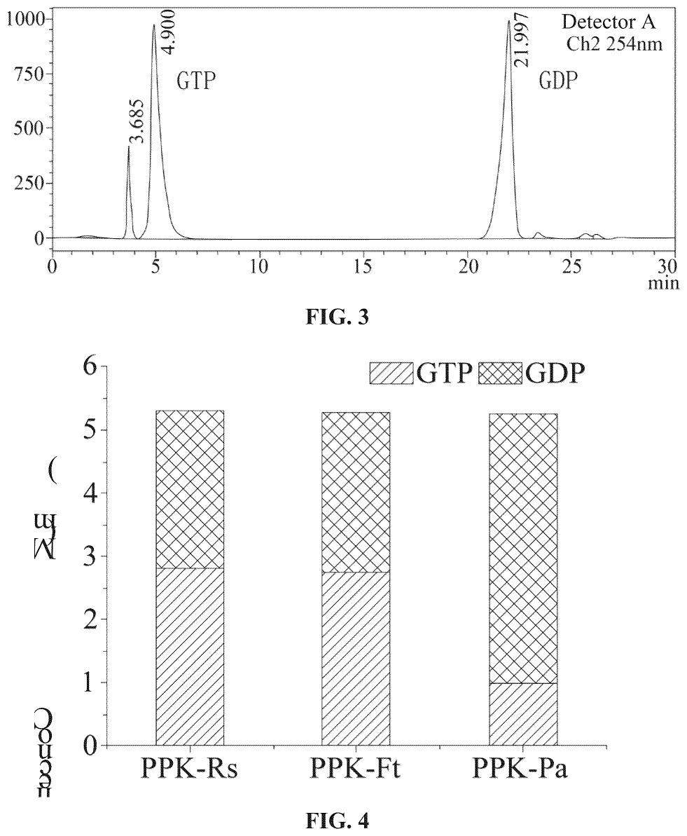

GTP and GDP standards of different concentrations were prepared and standard curves were plotted. HPLC showed that GTP and GDP had retention times of 22.0 min and 4.9 min, respectively, as shown in FIG. 3. The activities of PPK-Rs, PPK-Pa, and PPK-Ft obtained by HPLC were shown in FIG. 4. The results showed that after 1 h of reaction at 30° C., the PPK-Rs and PPK-Ft had similar abilities to regenerate GTP, and could regenerate more than 2.5 mM of GTP, with conversion rates of 56.2% and 55%, respectively; while the PPK-Pa could regenerate about 1 mM of GTP, with a conversion rate of 19.6%.

### Example 3: Use of PPKs to Regenerate GTP for Efficient Synthesis of GDP-L-Fucose and Fucosyllactose In Vitro

2′-FL was synthesized using a de novo synthesis route 2 in FIG. 1. The GDP-L-fucose de novo synthesis-enzyme is selected from the group consisting of polyphosphate-dependent glucomannokinase (PPGMK) derived from Arthrobacter species, phosphomannomutase (manB) derived from Streptomyces coelicolor, mannose-1-phosphate guanylytransferase (manC) derived from Escherichia coli, GDP-D-mannose-4,6-hydratase (gmd) derived from Pseudomonas aeruginosa PAO1, GDP-L-fucose synthase (wcaG) from Helicobacter pylori, and α-1,2-fucosyltransferase (FutC) from Helicobacter pylori were subjected to codon optimization and synthesis by GenScript as described in Example 1, and the recombinant bacteria obtained after plasmid transfection were expressed and purified, respectively.

The purified enzyme was used for the next reaction, where contents of the PPGMK, manB, manC, gmd, wcaG, FutC and PPK in the systhesis reaction system each were (0.1-2) mg/mL, and there were 20 mM mannose, 40 mM polyP6, 20 mM lactose, 20 mM Mg²⁺, 2 mM GTP, 5 mM NADPH, 20 mM sodium isocitrate, and 1 U/mL isocitrate dehydrogenase (regenerated NADPH). The reaction was conducted at 30° C. for 1 h to 48 h using 50 mM Tris-HCl (pH=8.0) as a buffer. The reaction product was detected by Shimadzu HPLC with a differential refractometer, a BIO-RAD organic acid analysis column (300×7.8 mm), 5 mM H₂SO₄ as a mobile phase, an isocratic elution program, a flow rate of 0.6 mL/min, and an injection volume of 10 μL.

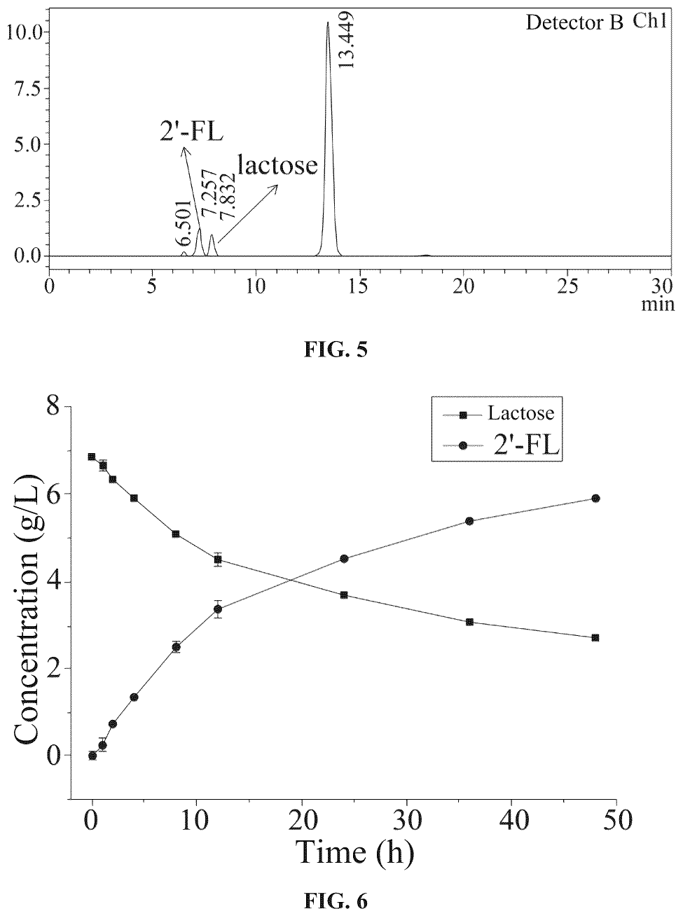

Lactose and 2′-FL standards with different concentrations were prepared and standard curves were plotted. HPLC showed that lactose and 2′-FL had retention times of about 7.83 min and 7.26 min, respectively, as shown in FIG. 5. A 2′-FL yield obtained by HPLC was shown in FIG. 6. With 20 mM (6.84 g/L) of lactose and 2 mM of GTP, an in vitro one-pot reaction was conducted for 48 h, and the 2′-FL yield reaches 5.93 g/L, and the reaction could still proceed after 48 h.

The above are only some preferred examples of the present disclosure, and the present disclosure is not limited to the contents of the examples. It should be pointed out that changes and improvements made by those skilled in the art without departing from the technical solution of the present disclosure should be within the protection scope of the present disclosure.

What is claimed is:

1. Polyphosphate kinases (PPKs) for efficient regeneration of guanosine triphosphate (GTP) and use thereof, wherein the PPKs have amino acid sequences being one selected from the group consisting of:
(1) amino acid sequences shown in SEQ ID NO: 1, SEQ ID NO: 2, and SEQ ID NO: 3; and
(2) amino acid sequences having a same function and obtained by subjecting amino acid sequences shown in SEQ ID NO: 1, SEQ ID NO: 2, and SEQ ID NO: 3 to modification comprising substitution, deletion, or addition of one or more amino acids.

2. PPK genes, wherein the PPK genes encode the PPKs according to claim 1.

3. A recombinant plasmid vector carrying the PPK genes according to claim 2.

4. The recombinant plasmid vector according to claim 3, wherein the recombinant plasmid vector comprises but is not limited to pET28a (+).

5. A recombinant microorganism expressing the PPKs according to claim 1.

6. The recombinant microorganism according to claim 5, wherein the recombinant microorganism is Escherichia coli BL21(DE3).

7. A method for regeneration of GTP as well as synthesis of guanosine diphosphate (GDP)-L-fucose or GDP-mannose and a derivative thereof, wherein the PPKs according to claim 1 are prepared using a polyphosphate (polyP).

8. The method according to claim 7, wherein the GDP-L-fucose is synthesized by in vitro multi-enzyme cascade catalysis using a GDP-L-fucose synthesis-related enzyme, the GDP-L-fucose and lactose are catalyzed to generate fucosyllactose using fucosyltransferase, and GTP regenerated by the PPKs is added to synthesize the GDP-L-fucose and the fucosyllactose.

9. The method according to claim 8, wherein a reaction system of the synthesis comprises 0.1 mg/mL to 2 mg/mL of a GDP-L-fucose de novo synthesis-related enzyme, 0.1 mg/mL to 2 mg/mL of the fucosyltransferase, 0.1 mg/mL to 2 mg/mL of the PPKs, 1 mM to 50 mM of mannose, 1 mM to 50 mM of lactose, 1 mM to 50 mM of the polyP, 1 mM to 10 mM of the GTP or GDP, and 1 mM to 50 mM of nicotinamide adenine dinucleotide phosphate (NADPH or NADP⁺); and the synthesis is conducted under a pH value of 6 to 10 at 20° C. to 40° C. for 1 h to 48 h.

10. The method according to claim 8, wherein a reaction system of the synthesis comprises 0.1 mg/mL to 2 mg/mL of a GDP-L-fucose salvage synthesis-related enzyme, 0.1 mg/mL to 2 mg/mL of the fucosyltransferase, 0.1 mg/mL to 2 mg/mL of the PPKs, 1 mM to 50 mM of fucose, 1 mM to 50 mM of lactose, 1 mM to 50 mM of the polyP, 1 mM to 10 mM of the GTP or GDP, and 1 mM to 10 mM of adenosine triphosphate (ATP) or adenosine diphosphate (ADP); and the synthesis is conducted under a pH value of 6 to 10 at 20° C. to 40° C. for 1 h to 48 h.


SEQUENCE LISTING (6 sequences)

SEQ ID NO: 1 - protein, 336 aa, Rhodobacter sphaeroides
  source 1..336: note = PPK
MAEDRAMPVMPPAADAAEAVPAAPTALPEEGPAGPEAPLQTLHGPRHFPAVDANAIRQAFEGGHYPYPRRLGRVVYEAEKARLQAELLKVQIWAQETGQKFVILMEGRDAAGKGGTIKRFMEHLNPRYARVVALTKPGERERGQWFFQRYIEHLPTAGEIVFFDRSWYNRAGVERVMGFCTPSEYLEFMRQAPELERMLVRSGIRLYKYWFSVTRDEQRARFLARETDPLKRWKLSPIDKASLDKWDDYTEAKEAMFFYTDTADAPWTIVKSNDKKRARLNCMRHFLSSLDYPGKDPEVVGVPDPLIVGRAAQVIGTAADILDSATPPALRKPRQG

SEQ ID NO: 2 - protein, 269 aa, Francisella tularensis
  source 1..269: note = PPK
MKVLSQEERQKLFLENIFPYKHKIPRNVYEKQKHYLQIELLKFQKWVKENNKKVLIIFEGRDAAGKGGTIKRMMEHLNPRGAKVIALEKPSEQERNQWYFQRYIEHLPSGGEIVLFDRSWYNRAGVERVMGFCTEREYFLFLEQAPQLEKMLVDSGTMIIKFWFSVSQQEQKNRFAARESHPLKQWKLSPIDKASLDKWDDYTEAKERMFIYTDKPYAPWVIVKSDDKKRARLNAIRYILNNVDYDNKDHEVAIPPDPLIVGTSSKIYK

SEQ ID NO: 3 - protein, 357 aa, Pseudomonas aeruginosa
  source 1..357: note = PPK
MSEEPTVSPPSPAQPAAQPAKPARPAARRAPRKPATRRPRVASPAQKAREEIQAISQKPVALQVASAPHGSSEDSTSASLPANYPYHTRMRRNEYEKAKHDLQIELLKVQSWVKETGQRVVVLFEGRDAAGKGGTIKRFMEHLNPRGARIVALEKPSSQEQGQWYFQRYIQHLPTAGEMVFFDRSWYNRAGVERVMGFCSPLQYLEFMRQAPELERMLTNSGILLFKYWFSVSREEQLRRFISRRDDPLKHWKLSPIDIKSLDKWDDYTAAKQAMFFHTDTADAPWTVIKSDDKKRARLNCIRHFLHSLDYPDKDRRIAHEPDPLLVGPASRVIEEDEKVYAEAAAAPGHANLDIPA

SEQ ID NO: 4 - DNA, 1008 bp, Rhodobacter sphaeroides
  source 1..1008: mol_type = other DNA; note = PPK gene
atggcagaagatagggctatgcccgtaatgccgcctgcggcggacgctgcggaggcggttccggcggcgccaaccgctctgccggaagagggcccagctggtccggaggcaccactccaaaccctgcatggtccgcgtcactttccggcggtcgatgcgaacgcgatccgtcaagcctttgaaggcggtcattatccgtatccgcgtcgcttaggccgtgtggtatatgaagcagagaaagcgcggctgcaagcggagctgctgaaggtgcagatttgggcacaagaaaccggtcagaagttcgtgatcctgatggagggccgtgacgccgcgggcaagggtggcaccatcaaaaggttcatggagcacttgaacccgcgttacgcacgcgtcgtggcattgaccaagccgggtgagcgcgaacgtggccagtggttttttcagcgttacattgaacacctgccgacggccggcgagatcgtattcttcgaccgttcctggtataaccgtgctggcgtggaacgtgttatgggtttctgcacgccgtctgaatatctggaatttatgcgccaggctccagagttggaacgtatgttggttcgttctggtattcgcctgtacaaatactggttcagcgttacccgtgacgagcagcgtgcgcgtttcctggcacgcgagactgacccgctgaagcgttggaagctgtccccgattgacaaggcatcgctggacaaatgggatgattacaccgaagcgaaagaagccatgtttttctacaccgacactgcggatgcgccgtggaccattgtgaagagcaatgataagaaacgtgcaaggttgaattgtatgagacactttctgagcagcttggactacccgggtaaagatccggaggttgtgggcgttccggatccgcttatcgtgggtcgcgctgcccaggttatcggtacggcggctgatatcctggacagcgctaccccgccggcgttacgcaaaccgcgccaaggt

SEQ ID NO: 5 - DNA, 807 bp, Francisella tularensis
  source 1..807: mol_type = other DNA; note = PPK gene
atgaaggtactatcacaagaggaaaggcagaaattgttccttgagaacattttcccgtataaacacaaaattccgcgcaacgtgtatgaaaaacaaaaacattatctgcagatcgaactgctgaagttccaaaagtgggttaaagagaataacaaaaaagtgttgatcatcttcgagggccgtgatgctgcgggtaaaggtggcaccattaaacgtatgatggaacacctgaacccgcgtggtgcaaaggtcatcgccctggagaagccgtctgaacaagagcgtaaccagtggtacttccagcgttatatcgagcacctgccgtccggtggtgaaatcgtcttgtttgaccgttcttggtacaacagagctggcgtggaacgtgttatgggtttttgcaccgagcgcgagtattttttattcttggagcaagcgccacagcttgagaagatgctggttgactccggcactatgattatcaaattctggtttagcgttagccagcaggagcaaaagaaccgttttgcggcgcgtgaaagccatccgctgaaacagtggaaactgagcccgattgataaggcgtcgctggataagtgggacgactacaccgaagcgaaagaacgcatgtttatctacaccgataagccgtacgctccgtgggttattgttaagtccgacgacaaaaagcgcgcgcgtctgaacgccatccgctacattctgaataatgtggactatgataataaggaccatgaagtggcaattcctccggatccgctgatcgtgggcacgagcagcaaaatctacaag

SEQ ID NO: 6 - DNA, 1071 bp, Pseudomonas aeruginosa
  source 1..1071: mol_type = other DNA; note = PPK gene
atgtcagaagagcctacagtatctcccccaagtccggctcaaccggcggcgcagccggctaagccggcgagaccggctgcacgccgtgcgccacgtaaaccagctacccgtcgtccgcgcgtggcgtcgccggcgcaaaaagcgagagaggagatccaagcaatttctcagaagcctgtagctctgcaggttgcgagcgcaccgcatggcagctccgaagacagcaccagcgcgtccctgccggcgaactatccgtaccatacgcgcatgcgtcgcaatgaatatgaaaaggccaagcacgatctgcaaattgagttgcttaaggtgcagagctgggttaaagaaaccggtcagcgtgtggtggttctgttcgaaggccgtgacgcggcgggcaagggtggtacaattaagcgctttatggaacacctgaacccgagaggcgcgcgtattgtggccttggagaagccgtcctctcaagagcagggtcaatggtacttccaacgttatatccagcacttgccaacggcgggcgaaatggtttttttcgatcgtagctggtacaatcgtgcgggtgttgaacgtgtcatgggcttctgcagccccctgcaatatctggagtttatgcgccaggctccggaattggagcgtatgttgaccaacagcggtatcctgctcttcaaatactggttcagcgtttcaagagaggagcagctgcgtcgtttcatctctcgtcgcgacgacccgctgaaacattggaagctgagcccgattgatatcaagtccttagacaagtgggacgactacactgcggccaaacaggcaatgttttttcacaccgataccgcggatgcgccgtggaccgtgattaaatctgatgacaaaaaacgcgcacgcctgaattgtattcgtcattttctccacagcctggattacccggataaagatcgccgtatcgcccacgaaccggacccgttgctggttggtccggcctcccgtgtcatcgaggaagatgaaaaagtctatgcagaggcagcggcagctccgggtcatgccaacctggacatcccggcg